(19) United States
(12) Patent Application Publication
(10) Pub. No.: US 2010/0189766 A1
(43) Pub. Date: Jul. 29, 2010

(54) SUBSTANCE DELIVERING PUNCTUM IMPLANTS AND METHODS

(75) Inventors: Deepank Utkhede, Surrey (CA); Wendy Monk, Surrey (CA)

Correspondence Address: Pillsbury Winthrop Shaw Pittman LLP (QLT), P.O. Box 10500, McLean, VA, 22102, US

(73) Assignee: QLT PLUG DELIVERY, INC., Menlo Park, CA (US)

(21) Appl. No.: 12/643,502

(22) Filed: Dec. 21, 2009

Related U.S. Application Data
(60) Provisional application No. 61/139,456, filed on Dec. 19, 2008.

(51) Int. Cl. A61F 2/00; A61P 27/06

(52) U.S. Cl. 424/427

(57) ABSTRACT

Substance delivering punctum plug devices and related methods for treating disorders and diseases of the eye. Some embodiments of the device maximize dissolution of the substance in tears and/or other fluid(s) that distribute to the anterior surface of the eye so as to maximize delivery of the substance to or through the cornea or anterior surface of the eye ball while minimizing loss of substance through other routes.

### RELATED APPLICATIONS

This patent application claims priority to U.S. Provisional Patent Application No. 61/139,456 filed Dec. 19, 2008, the entire disclosure of which is expressly incorporated herein by reference.

### FIELD OF THE INVENTION

The present invention relates generally to medical devices and methods and more particularly to implantable devices for delivering therapeutic or diagnostic substances and related methods for manufacturing and using such devices.

### BACKGROUND OF THE INVENTION

The naso-lacrimal system is a series of openings and ducts which form a miniature drainage network through which tears may drain from the eye into the nasal cavity. Tears which collect on the surface of the eye flow along the edges of the eye lids toward the nose where they pass through small openings known as puncta (i.e., the superior punctum and inferior punctum) and then into ducts known as canaliculi. The tears flow through the canaliculi into a structure known as the lacrimal sac. Tears which accumulate in the lacrimal sac then drain from the lacrimal sac, through a duct known as the naso-lacrimal duct, into the nasal cavity.

In adults, each punctum is about 0.3 mm in diameter. The superior and inferior puncta are located in the medial aspects of the upper and lower eyelid margins, respectively. Each punctum sits on top of a raised bump known as the papilla lacrimalis.

After passing trough each punctum the tears flow into an initial vertical segment of the canaliculus. Such vertical segment is typically about 2 mm in length. Then the tears flow through a horizontal segment of the canaliculus that is about 8 mm in length. The angle between the vertical and horizontal segments of each canaliculus is approximately 90 degrees. In most individuals, the horizontal segments of the canaliculi merge to from a common canaliculus which then leads into the lacrimal sac.

Punctum plugs are small plugs that are insertable into the puncta. Punctum plugs were initially used to treat dry eye syndrome by blocking the outflow of tears from the eye and, thus, increasing the thickness of the tear film that covers the eye. More recently, drug-eluting punctum plugs have been proposed for use as drug delivery implants for treatment of certain disorders of the eye. For example, a clinical study has demonstrated that substance eluting punctum plugs may be used to deliver dosages of latanoprost, a prostaglandin analog, to the anterior segment of the human eye for the treatment of open angle glaucoma or ocular hypertension. EyeNet Magazine, News in Review, page 23 (October 2008).

Drugs used to treat glaucoma as well as drugs used to treat corneal disorders are most effective when delivered topically to the anterior surface of the eye.

Accordingly, there is a need in the art for improved punctum plugs that maximize delivery of drugs and/or other substances to the corneal surface.

### SUMMARY OF THE INVENTION

The present invention provides implantable substance delivery devices that are implantable in the puncta of the eyes of human or animal subjects and useable to deliver therapeutic or diagnostic substance(s) to the eye or other areas of the body. Additionally, the present invention provides methods for treating various disorders using the implantable substance delivery devices of the present invention.

In accordance with one aspect of the present invention, there are provided substance delivery devices that are implantable in a punctum of the eye of a human or animal subject. These devices generally comprise (A) a punctum plug that has a plug body and a substance insert-receiving cavity formed in the plug body and (B) a substance insert that consists of or comprises a substance eluting core having a distal end and a proximal end, wherein (C) the substance insert is positioned in the substance insert-receiving cavity of the punctum plug such that, when the device is implanted in the punctum of an eye, a first tear-eluting location at a proximal end of the substance eluting core will be exposed to tears and/or other fluid(s) that distribute to the anterior surface of the eye so that the substance will elute from that first tear-eluting location into tears and/or other fluid(s) that distribute to the anterior surface of the eye and at least one additional tear-eluting location on the substance eluting core will also be exposed to tears and/or other fluid(s) that distribute to the anterior surface of the eye so that the substance will also elute from said at least one additional tear-eluting location into tears and/or other fluid(s) that distribute to the anterior surface of the eye. In some embodiments, one or more channel(s) are formed to allow tears and/or other fluid(s) to flow into an area that is adjacent to the substance core, thereby creating the additional tear-eluting location. In some embodiments that include such channel(s), all or part of the device may be formed of elastic or flexible materials that allow the channel(s) to flex or expand/contract in response to movements of facial muscles, thereby creating a pumping-like effect facilitating movement (e.g., turn over) of tears and/or other fluid(s) into and out of the channel(s). In some embodiments a portion (e.g., a distal end) of the substance core may be rendered substantially impervious to the substance so that little or no substance will pass through that portion of the substance core while the substance remains free to elute from the desired tear-eluting locations. Also, in some embodiments, all or part of the punctum plug body may be rendered substantially impervious to the substance to deter or eliminate unwanted diffusion of the substance through the punctum plug while allowing the substance to elute from the desired teat-eluting locations.

In accordance with another aspect of the present invention, there are provided methods for using implantable substance delivery devices of the foregoing character. In these methods, the substance delivery device is implanted in the punctum of an eye of a human or non-human animal subject to deliver a substance for the diagnosis or treatment of a disease or disorder or for experimental purposes, such as the creation of an experimental animal model wherein the implanted substance delivery device is used to administer a substance that creates a pathological state for purposes of laboratory study and/or testing of possible treatments. The devices and methods of the present invention are particularly suited for delivery of substances to the anterior surface of the eye via tears and/or other fluid(s) that distribute to the anterior surface of the eye. Thus, the devices and methods of the present invention may have particular utility in treating diseases and disorders that affect the cornea, conjunctiva, sclera, anterior chamber, anterior chamber angle, trabecular meshwork, Schlemm's canal, collector channels emanating from Schlemm's canal or other anatomical structures in the eye to which a therapeutically effective amount of a substance that has been administered topically to the anterior surface of the eye will distribute.

Further aspects, objects, applications, elements, details and particulars of the present invention will be understood by those of skill in the relevant art upon reading of the detailed description and examples set forth herebelow.

### BRIEF DESCRIPTION OF THE DRAWINGS

FIG. 1 is a diagram of a human eye and associated lacrimal drainage system with a substance-delivery device of the present invention implanted in the inferior or lower punctum.

FIG. 1A is an enlarged view of region 1A of FIG. 1.

FIG. 1B is a diagram of a tear film covering the cornea of the eye shown in FIG. 1.

FIG. 2 is an exploded side view of a first embodiment of a substance delivery device of the present invention.

FIG. 2A is a perspective view of the fully assembled substance insert component of the substance delivery device of FIG. 2.

FIG. 2B is a partial top view of the substance delivery device of FIG. 2.

FIG. 3 is an exploded side view of a second embodiment of a substance delivery device of the present invention.

FIG. 3A is a perspective view of the fully assembled substance insert component of the substance delivery device of FIG. 3.

FIG. 3B is a partial top view of the substance delivery device of FIG. 3.

FIG. 4 is an exploded side view of a third embodiment of a substance delivery device of the present invention.

FIG. 4A is a top view of the punctum plug component of the substance delivery device of FIG. 4.

FIG. 4B is a perspective view of the fully assembled substance insert component of the substance delivery device of FIG. 4.

FIG. 4C is a partial top view of the substance delivery device of FIG. 4.

FIG. 5 is an exploded side view of a fourth embodiment of a substance delivery device of the present invention.

FIG. 5A is a partial top view of the substance delivery device of FIG. 5.

FIG. 6 is an exploded side view of a fifth embodiment of a substance delivery device of the present invention.

FIG. 6A is a partially-exploded/partially assembled perspective view of the substance insert component of the substance delivery device of FIG. 6.

FIG. 6B is a fully assembled perspective view of the substance insert component of the substance delivery device of FIG. 6.

FIG. 7 is a side, partially cut-away view of an alternative punctum plug component useable in substance delivery devices of the present invention wherein a coating (shown partially cut away) is provided on a region of the punctum plug that surrounds the substance insert to prevent or deter passage of the substance through that portion of the punctum plug.

FIGS. 8A through 8C are perspective views of alternative configurations of the punctum plug component of substance delivery devices of the present invention.

### DETAILED DESCRIPTION AND EXAMPLES

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

The entire disclosure of each of the following co-pending United States and PCT International patent applications are expressly incorporated herein by reference:

| Application Ser. | Publication | | |
|---|---|---|---|
| No. | Number | Filing Date | Country |
| 12/231,989 | 2009/0104248 | Sep. 8, 2008 | United States |
| 12/231,986 | 2009/0104243 | Sep. 5, 2008 | United States |
| 12/378,710 | 2009/0264861 | Feb. 17, 2009 | United States |
| 12/463,279 | 2009/0280158 | May 8, 2009 | United States |
| 12/432,553 | N/A | Apr. 29, 2009 | United States |
| PCT/US2009/002611 | WO 2009/134371 | Apr. 29, 2009 | PCT |
| 12/231,986 | 2009/0104243 | Sep. 5, 2008 | United States |

Definitions: As used herein, the following words and/or phrases shall be defined as follows:
The term “substance” includes diagnostic, therapeutic, nutritional and cosmetic substances such as, but not limited to; chemicals, drugs, therapeutic agents, diagnostic agents, biologics, radioactive agents, contrast agents, dyes, lubricants and nutrients, including but not limited to those listed in any of applications listed in the above-set-forth table and expressly incorporated herein by reference.
The term “subject” includes human and other animal subjects, including human and animal patients who receive therapeutic, preventative or diagnostic treatment as well as human or animal experimental subjects to whom a particular substance is administered for experimental purposes, to study or observe effects of the substance or to induce a disease or disorder as in an animal model for such disease or disorder.
The term “other fluid(s)” includes body fluid or fluids other than tears (e.g., tissue or interstitial fluids, mucoid secretions, lipid secretions, etc.) as well as fluids of exogenous origin (e.g., artificial tears, medicated or non-medicated eye drops, eye-wash fluids, lavage fluids, etc.).

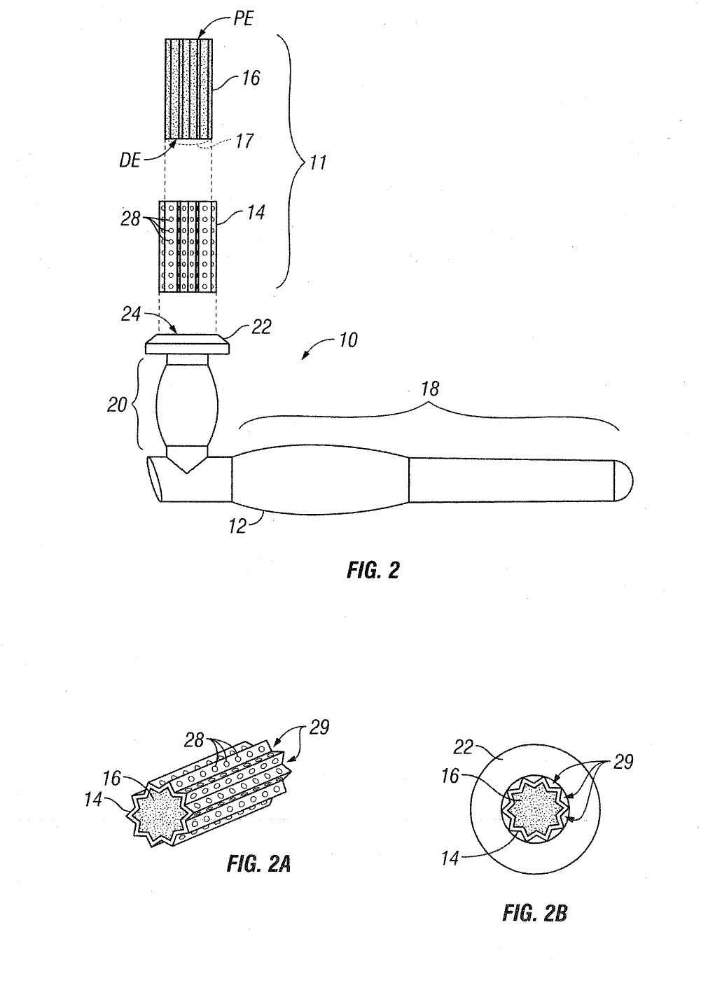

The accompanying drawings and the following detailed description and examples are intended to show and describe some, but not necessarily all, examples or embodiments of the invention. The described examples and embodiments are to be considered in all respects as illustrative and not restrictive. The drawings of FIGS. 2-6 are to be considered as illustrative with respect to the components of the devices and are not intended as an illustration of process of making those devices. The contents of this detailed description and the accompanying drawings do not limit the scope of the invention in any way.

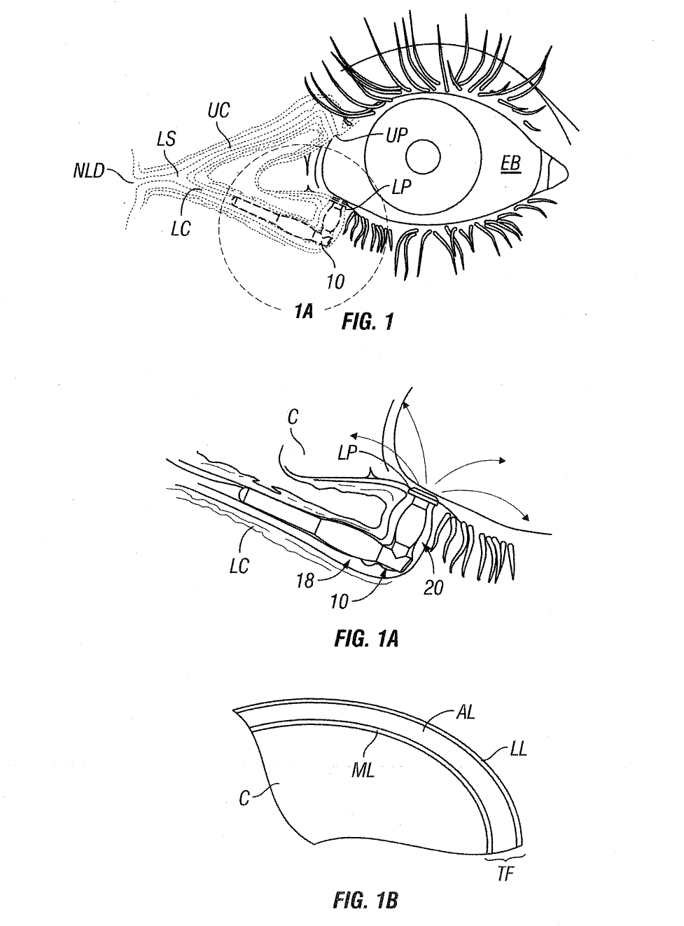

FIG. 1 is a diagram of a human eye anatomy and the associated lacrimal duct system. For purposes of the present discussion it will be sufficient to focus on the latter which consists of the upper and lower lacrimal ducts known as the upper canalicula UC and lower canalicula LC, and the tear or lacrimal sac LS. The upper and lower canaliculae UC, LC each terminate in a superior or upper punctal aperture UP and an inferior or lower punctal aperture LP, respectively. The punctal apertures UP, LP are round or slightly ovoid openings approximately 0.3 mm in size and surrounded by a fairly dense, relatively avascular connective ring of tissue about 1 mm in depth. Each of the punctal apertures UP, LP leads into a generally vertical segment of the respective canaliculus UC or LC. Each canaliculus UC, LC is a tube of approximately 0.5 mm diameter lined by stratified squamous epithelium surrounded by elastic tissue which allows the canaliculus to be dilated to about three times its normal size. The tears flow through the canaliculi UC and LC and into the lacrimal sac LS. Tears which accumulate in the lacrimal sac LS then drain from the lacrimal sac LS, through the naso-lacrimal duct NLD and into the nasal cavity.

In FIG. 1, one embodiment of a substance delivery device 10 of the present invention is implanted in the lower punctal aperture LP and lower canaliculus LC, as shown.

FIG. 1B shows a typical tear film TF on the anterior surface of the cornea C of a human eye. This tear film TF consists of three layers, namely an inner mucoid layer ML, an outer lipid layer LL and an aqueous layer AL therebetween.

FIGS. 2-2B show a first embodiment of a substance delivery device 10 of the present invention. In this embodiment, the substance delivery device 10 comprises a punctum plug component 12 and a substance insert component 11. The punctum plug 12 has an elongate body portion 18 and a substance insert receiving portion 20. The punctum plug may be formed of any suitable non-biodegradable, partially biodegradable or fully biodegradable materials(s) as described in the applications listed in the above-set-forth table and expressly incorporated herein by reference. Examples of materials of which the punctum plug 12 may be formed include but are not limited to elastomers, silicones, hydrophilic polymers, hydrophilic silicones, polyurethanes, acrylics, polyvinyl alcohol, hydrogels, polyurethane hydrogels, solid hydrogels, silicone/polyurethane copolymers, silicone/poly(ethylene glycol copolymers, silicone/2 hydroxyethyl methacrylate copolymers (HCMA) and others. A substance insert receiving cavity 24 is formed within the substance insert receiving portion 20 and a flange 22 optionally extends around the mouth or opening of the substance insert receiving cavity 24. The substance insert 11 comprises a sheath 14 and a substance core 16. The substance core 16 of this embodiment or any other embodiment described herein comprises a matrix material such as a polymer or other suitable material that is combined or loaded with a therapeutic or diagnostic substance. Specific matrix materials, therapeutic/diagnostic substances and methods for making the substance core 16 are fully described in the applications listed in the above-set-forth table and expressly incorporated herein by reference. One specific example of a material of which the matrix of the substance core 16 may be formed is silicone elastomer (e.g., Nusil MED6385, Nusil Technology, L.L.C., Carpinteria, Calif.). The sheath 14 has a lumen into which the substance core 16 is inserted. In this embodiment, the side wall of the sheath 14 is corrugated such that elongate grooves or channels 29 are formed in the outer surface of the sheath 14. The sheath 14 of this embodiment and in the other embodiments described below is formed of material that is substantially impervious to the substance (e.g., a polyimid, polyethylene or polyethylene terephthalate (PET)) but apertures 28 are formed in the side wall of the sheath 14 along each channel 29. As seen in FIGS. 2A and 2B, the substance core is positioned within the lumen of the sheath 14 to from the substance insert 11. The substance insert 11 is then inserted, distal end first, into the substance insert receiving cavity 24 of the punctum plug 12. As seen in FIGS. 1 and 1A, the device 10 is inserted through the inferior or lower punctum LP such that the elongate body portion 18 of the punctum plug 12 resides in the horizontal portion of the inferior or lower canalicula LC and the substance insert receiving portion 20 resides within the vertical portion of the inferior or lower canalicula LC, with its flange 22 extending around the inferior or lower punctal aperture LP. Tears and/or other fluid(s) may pass into the channels 29 and substance may elute through apertures 28 into tears and/or other fluid(s) that have entered the channels 29. Also, the proximal end PE of the substance core 16 is exposed to tears and/or other fluid(s) so that drug may also elute from the proximal end of the substance core 16 into tears and/or other fluid(s) within the eye. Optionally, the device may be constructed of flexible materials such that the channels 29 may flex (e.g., compress and decompress) in response to routine facial muscle movements (e.g., blinking, opening and closing of the eyes, squinting, etc.) thereby creating a pumping action that will move tears and/or other fluid(s) into and out of the channels, thus increasing turn-over of tears and/or other fluid(s) within the channels and increasing the concentration of substance within the tear film TF and/or other fluid(s) covering the cornea C or anterior surface of the eye. This may be accomplished by forming all or part of the device 10 of material(s) that are sufficiently flexible to cause the channels 29 to flex or compress and decompress in response to routine movements of muscles of the face. Examples of such materials include but are not limited to silicone elastomers, hydrogels and other elastomers including hydrophilic polymers of the type typically used in the construction of soft contact lenses. Also, optionally, all or part of the distal end of the substance insert 11 may be rendered substantially impervious to the substance so that substance will not be lost through the distal end of the substance core 11. This may be accomplished, for example, by placing a seal 17 (e.g., a substance impervious cap or coating) on the distal end DE of the substance core 16. Such seal 17, in this embodiment or any other embodiment described herein, may be formed of a cyanoacrylate (e.g., a UV curable cyanoacrylate such as Loctite™ 4305, Henkel Corporation, Dusseldorf, Germany) or other suitable materials that is substantially impervious to the substance, such as for example parylene (poly-xylylene polymers), metallic coatings (e.g., silver-based coating which also provides antimicrobial activity), polyimides, impervious silicones, thermoplastics (like polyurethane, polyvinyl chloride), polytetrafluoroethylene (PTFE) or other minimally permeable polymers.

In FIGS. 2-2B, the sheath 14 is shown with a corrugated side wall such that the inner luminal wall has grooves as well as the outer wall surface. In this embodiment, the substance core 16 may be extruded, die cut, pressure formed or otherwise shaped into a corresponding shape (e.g., a star-like shape) so that, when inserted into the sheath 14, it will substantially fill the lumen of the sheath as seen in the drawings. It is to be appreciated, however, that alternative modes of constructing the sheath 14 are possible. For example, the longitudinal grooves may be extruded or cut only into the outer surface of the sheath 14 and its inner lumen may have a round, non-grooved inner wall. In such alternative embodiment, a substance core 16 having a simple cylindrical shape may be inserted into the lumen of the sheath 14 so as to substantially fill that lumen.

The components of this device 10 as well as the other embodiments 10a, 10b, 10c and 10d described herein, may be formed of any suitable materials. In some embodiments the device may be biodegradable. In other embodiments, it may be non-biodegradable. Examples of materials that may be used in the construction of the punctum plug 12, sheath 14 and substance core 16 as well as specific drugs and other substances that may be contained in the substance core 16 are described in the applications listed in the above-set-forth table and expressly incorporated herein by reference.

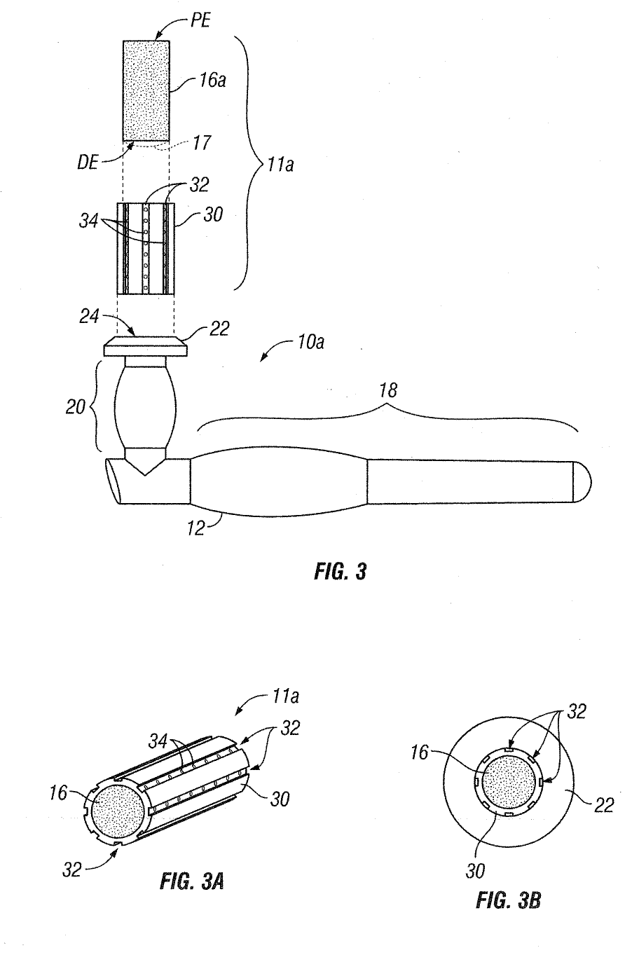

FIGS. 3-3B show a second embodiment of a substance delivery device 10a of the present invention. In this embodiment, the punctum plug component 12 that is the same as that shown in FIG. 2 and described above. However, in this embodiment, the substance insert 11a comprises sheath 30 (e.g., a tube) that has a round inner luminal wall in combination with a cylindrical substance core 16a. As described above with respect to the substance core 16 of the first embodiment, this substance core 16a may comprise a polymer matrix (e.g., a silicone elastomer) that is combined or loaded with a therapeutic or diagnostic substance. Longitudinal channels 32 in the form of rectangular grooves are cut (e.g., laser cut), extruded or otherwise formed in only the outer surface of the sheath 30, allowing its inner lumen wall to remain substantially round in cross section. The sheath 30 is formed of material that is substantially impervious to the substance (e.g., polyimid, polyethylene, PET, etc.). Apertures 34 are formed through the wall of the sheath 30 within each longitudinal channel 32. As seen in FIGS. 3A and 3B, the cylindrical substance core 16a is inserted into the round lumen of the sheath 30 to from the substance insert 11a. The substance insert 11a is then inserted, distal end first, into the substance insert receiving cavity 24 of the punctum plug 12. This device 10a may be inserted through the inferior or lower punctum LP such that the elongate body portion 18 of the punctum plug 12 resides in the horizontal portion of the inferior or lower canalicula LC and the substance insert receiving portion 20 resides within the vertical portion of the inferior or lower canalicula LC, with its flange 22 extending around the inferior or lower punctal aperture LP in the same manner as shown in FIGS. 1 and 1A. Tears and/or other fluid(s) may then pass into the channels 32 and substance may elute through apertures 34 into tears and/or other fluid(s) that have entered the channels 32. Also, as in the other embodiments, the proximal end PE of the substance core 16a is uncovered and exposed to tears and/or other fluid(s) so that drug may also elute from the proximal end of the substance core 16a into tears and/or other fluid(s) within the eye. Optionally, the device may be constructed of flexible materials such that the channels 32 may flex (e.g., compress and decompress) in response to routine facial muscle movements (e.g., blinking, opening and closing of the eyes, squinting, etc.) thereby creating a pumping action that will move tears and/or other fluid(s) into and out of the channels 32, thus increasing turn-over or exchange of tears and/or other fluid(s)s within the channels and increasing the concentration of substance within the tear film TF that covers the cornea C of the eye. Also, optionally, all or part of the distal end of the substance insert 11a may be rendered substantially impervious to the substance so that substance will not be lost through the distal end of the substance core 11a. This may be accomplished, for example, by the presence of an optional seal 17 (e.g., a substance impervious cap or coating) as described above, on the distal end DE of the substance core 16a.

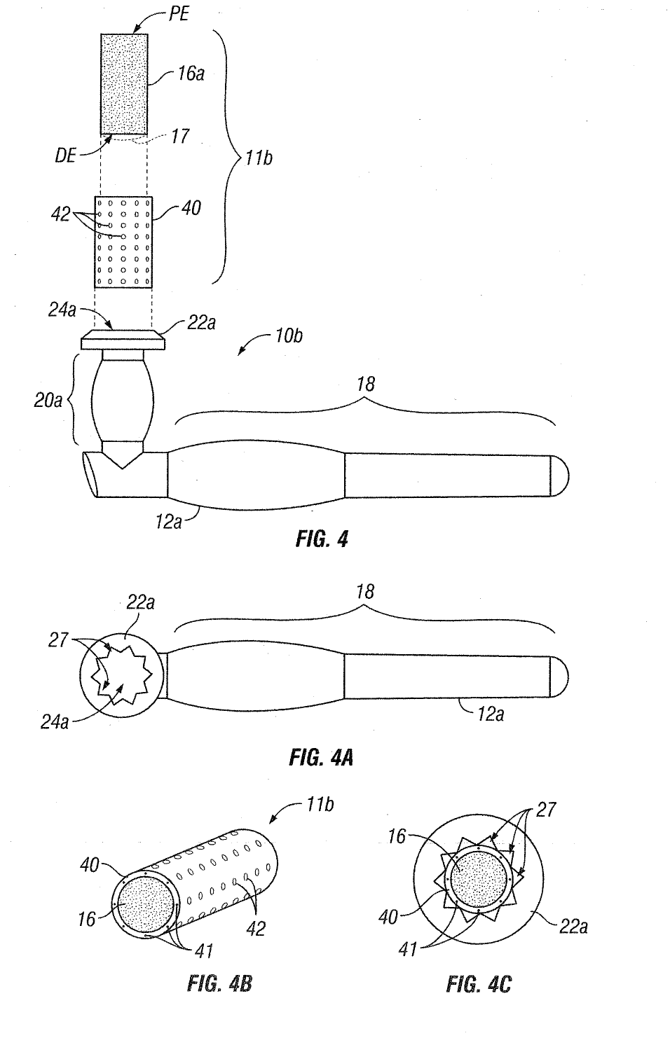

FIGS. 4-4C show a third embodiment of a substance delivery device 10b of the present invention. In this device 10b, the punctum plug 12a differs from the punctum plug 12 used in the embodiments of FIGS. 2 and 3 in that the substance insert-receiving portion 20a has a substance insert receiving cavity 24a that is polygonal in cross section rather than round in cross section. The substance insert 11b comprises a cylindrical substance-impervious sheath 40 having apertures 42 formed through its side wall and a cylindrical substance insert 16a that is positioned within the cylindrical sheath 40. As explained above, a seal 17 (e.g., a substance-impervious cap or substance-impervious coating) may optionally be provided on the distal end DE of the substance insert 16a to prevent the substance from eluting through the distal end DE of the substance insert 16a. As seen in FIG. 4C, when the cylindrical substance insert 11b is inserted into the polygonal substance insert-receiving cavity 24a, channels 27 will remain open such that tears and/or other fluid(s) may flow into the channels 27. The apertures 42 are preferably arranged in rows and the substance insert 11b is rotationally oriented within the substance insert-receiving cavity 24a such that the rows of apertures 42 reside adjacent to the channels 27 such that substance from the substance core 16a may elute through the apertures and into tears and/or other fluid(s) that enter the channels 27. To facilitate such precise rotational orientation of the substance insert 11b to align the apertures 42 with the channels 27, markers 41 may be provided on the proximal end of the substance insert 11a to indicate the position of some or all of the rows of apertures 42. Such markers 41 may then be used during assembly of the device to ensure that the apertures 42 reside adjacent to the channels 27, thereby facilitating elution of the substance into tears and/or other fluid(s) within the channels 27. As in the other embodiments, some or all of the wall of the substance insert receiving portion 20a of the punctum plug 12a may be constructed of flexible materials as described above to allow the walls of the channels 27 to flex (e.g., compress and decompress) in response to routine facial muscle movements (e.g., blinking, opening and closing of the eyes, squinting, etc.) thereby creating a pumping action that will move tears and/or other fluid(s) into and out of the channels 27, thus increasing turn-over or exchange of tears and/or other fluid(s) within the channels and increasing the concentration of substance within the tear film TF that covers the cornea C of the eye. Also, it is to be appreciated that various alternative designs of the embodiment may be devices wherein, rather than being polygonal in cross section, the wall of the substance insert receiving cavity 24a may be ribbed, grooved, oval or otherwise provided with surface disruptions, spacers or stand-offs so that one or more channels or spaces will exist between the outer surface of the substance insert 11b and the inner side wall of the substance insert receiving cavity 24a.

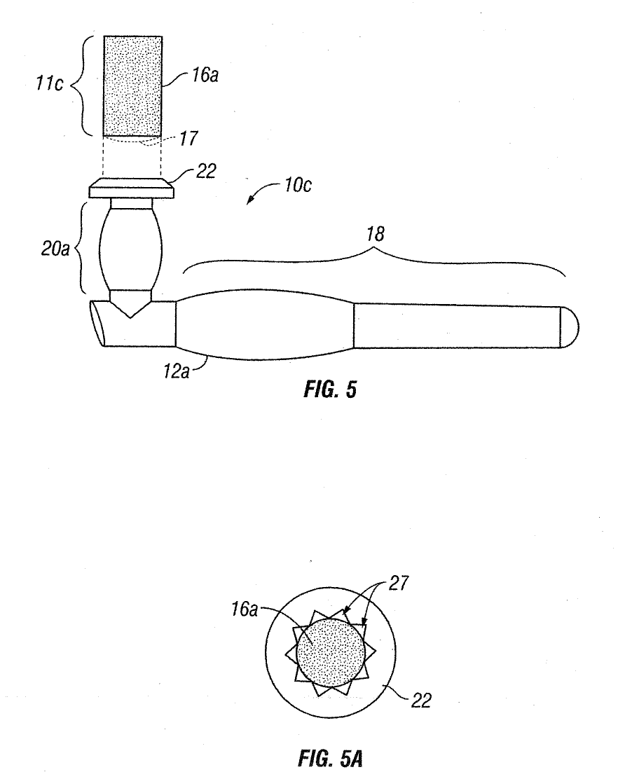

In some applications, the sheath portions 14, 30, 40 of the above-described substance inserts 11, 11a, 11b may be eliminated and the substance insert 11, 11a, 11b may consist of or consist substantially of the substance core 16, 16a alone. In some such embodiments, the desired grooves, depressions or other channels may be cut or formed directly in the outer surface of the substance core 16, 16a or in the adjacent inner wall of the substance insert receiving cavity 24, 24a of the punctum plug 12, 12a. This concept is illustrated in a fourth embodiment of a substance delivery device 10c shown in FIGS. 5-5A. In this embodiment, the punctum plug 12a of FIG. 4 is used in conjunction with a substance insert 11c that consists only of substance core 16a, without any surrounding sheath. As explained above, a seal 17 (e.g., a substance-impervious cap or substance-impervious coating) may optionally be provided on the distal end DE of the substance core 16a to prevent the substance from eluting through the distal end DE of the substance core 16a. As seen in FIG. 5A, when the cylindrical substance core 16a is inserted into the polygonal substance insert-receiving cavity 24a, channels 27 will remain open such that tears and/or other fluid(s) may flow into the channels 27 and drug from the unsheathed substance core 16a may elute into tears and/or other fluid(s) that enter the channels 27.

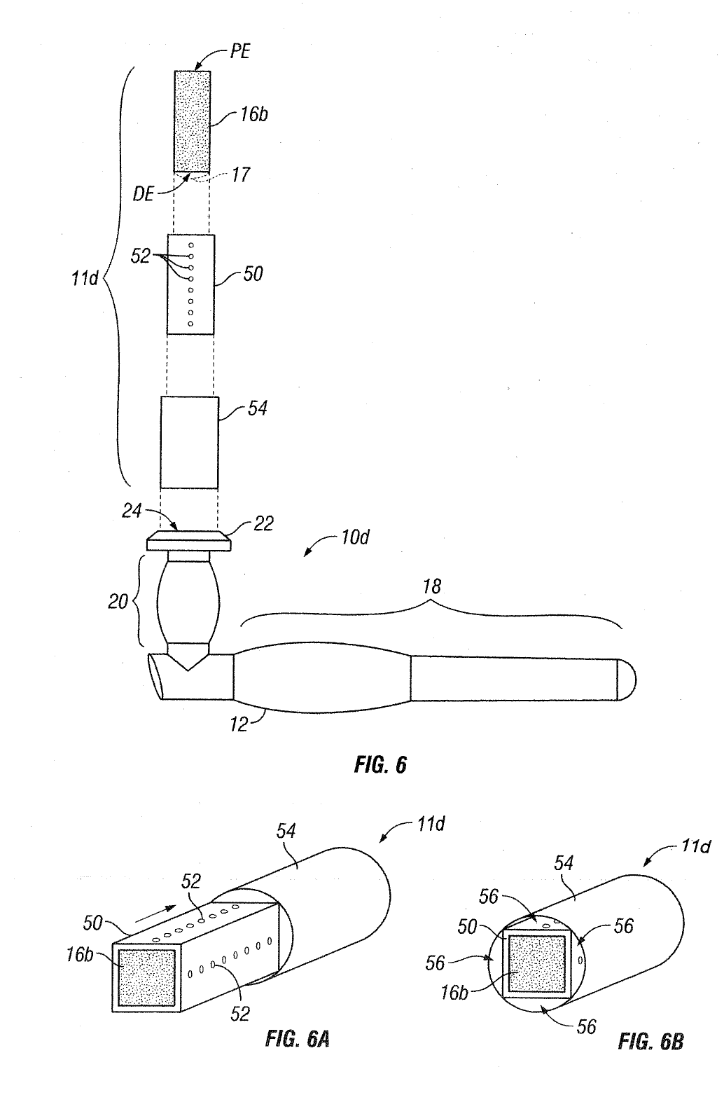

In some embodiments, the substance insert may incorporate multiple sheaths, with tear/fluid flow channel(s) being formed between the sheaths. For example, FIGS. 6-6B show a fifth embodiment of a substance delivery device 10d of the present invention. In this device 10d, the punctum plug 12 is the same as that shown in FIGS. 2 and 3 and described above and the substance insert 11d comprises a substance core 16b that is square in cross section, an inner sheath 50 that is square in cross section and an outer sheath 54 that is round in cross section. As described above with respect to the substance cores 16, 16a of other embodiments, this substance core 16b is formed of a matrix material combined or loaded with the desired therapeutic or diagnostic substance. The inner sheath 50 and outer sheath 52 are formed of material(s) that is/are substantially impervious to the substance (e.g., polyimid). Apertures 52 are formed in the side walls of the inner sheath 50, as shown. The substance core 16b is placed within and substantially fills the lumen of the inner sheath 50. The inner sheath 50 (with the substance core 16b positioned therein) is inserted into the lumen of the outer sheath 54, thereby forming the substance insert 11d for this embodiment. Because the inner sheath 50 is square in cross section and the outer sheath 54 is round in cross section, open channels 56 extend along the sides of the inner sheath 50, between the outer surface of the inner sheath 50 and the inner luminal surface of the outer sheath 54. The substance insert 11d is inserted within the substance insert-receiving cavity 24 of the punctum plug 12. The proximal end PE of the substance core 16b remains exposed such that, when implanted in the punctum, substance may elute from the proximal end of the substance core 16b into tears and/or other fluid(s) that accumulate in the area of the punctum. Additionally, when so implanted, tears and/or other fluid(s) may enter channels 56 and substance may elute from the substance core 16b, through apertures 52 and into tears and/or other fluid(s) that have entered the channels 56. As described above in relation to the other embodiments, some or all of the wall of the substance insert receiving portion 20 of the punctum plug 12, some or all of the outer sheath 54 and/or some or all of the inner sheath 50 may be constructed of flexible materials to allow the walls of the channels 56 to flex (e.g., compress and decompress) in response to routine facial muscle movements (e.g., blinking, opening and closing of the eyes, squinting, etc.) thereby creating a pumping action that will move tears and/or other fluid(s) into and out of the channels 27. Such turn-over or exchange of tears and/or other fluid(s) within the channels will result in a greater concentration of substance within the tear film TF that covers the cornea C of the eye.

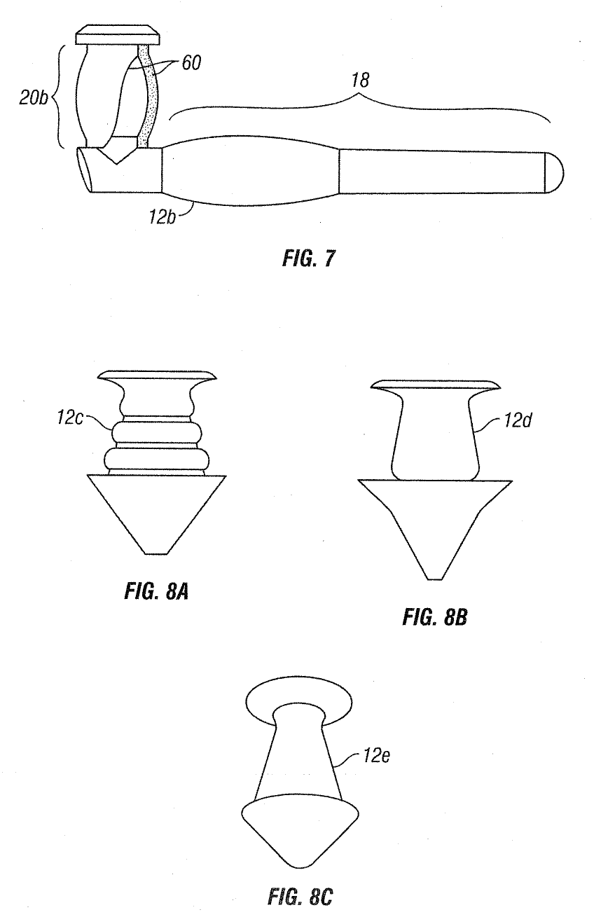

In applications where it is desired to deliver the therapeutic or diagnostic substance to or through the cornea or the eye it is desirable to cause the substance to become dissolved in the tear film TF which covers the cornea of the eye, while deterring or minimizing untoward loss of substance through other routes, such as through direct absorption into tissues surrounding the punctum and/or canaliculus LC in which the device is implanted (referred to generally herein as “pericanalicular tissue absorption”) or by drainage through the naso-lacrimal system into the nose. One way to limit or deter such loss of substance is to render all or part of the punctum plug 12, 12a impervious to the substance so that the substance cannot diffuse or pass through the wall of the punctum plug 12, 12a. This may be particularly important in embodiments where the punctum plug 12, 12a is formed of silicone or other material through which the substance can pass. In this regard, FIG. 7 shows a modified punctum plug 12b that has an elongate body portion 18 and a substance insert receiving portion 20b. The substance insert receiving portion 20b may have the same configuration as either of those substance insert receiving portions 20, 20a described above. However, in this embodiment, a coating 60 has been applied to the outer surface of the side wall of the substance insert receiving portion 20b. Such coating may also be applied to other portions of the punctum plug 12b or to the entire punctum plug 12b. This coating 60 is a material that is impervious to whatever substance is to be eluted from a substance insert positioned within the substance insert receiving portion 20b of this punctum plug. This will prevent the substance from passing through the side wall of the substance insert receiving portion 20b (or any other portions of the punctum plug 12b on which the coating 60 is present). The presence of this coating 60 may limit the potential for the substance to undergo pericanalicular tissue absorption or drainage though the naso-lacrimal system in applications where such modes of substance absorption/loss are not desirable. This coating 60 may also maximize the amount of substance that becomes dispersed or dissolved in the tear film TF and thus delivered, via the tear film, to the anterior surface of the eye. In particular, Applicants have determined that in embodiments where the punctum plug 12b is formed of silicone and the substance insert contains latanoprost for treatment of glaucoma, some of the latanoprost will diffuse or pass through the wall of the punctum plug 12 and thus may be lost through pericanalicular tissue absorption or drainage through the naso-lacrimal system. This is problematic because latanoprost is most effective for the treatment of glaucoma when delivered topically to the cornea of the eye, such that it is absorbed through the cornea and into the anterior chamber of the eye. However, when a substance-impervious coating 60 is applied to the outer surface of the substance insert receiving portion 20b and or other portions of the punctum plug 12b, the loss of latanoprost by diffusion or passage through the punctum plug 12b is substantially prevented. Such coating 60 may be formed of any suitable material, for example a cyanoacrylate (e.g., a UV curable cyanoacrylate such as Loctite™ 4305, Henkel Corporation, Dusseldorf, Germany) or other suitable material that is substantially impervious to the substance, such as, for example; parylene (poly-xylylene polymers), metallic coatings (e.g., silver-based coating which also provides antimicrobial activity), polyimides, impervious silicones, thermoplastics (like polyurethane, polyvinyl chloride), polytetrafluoroethylene (PTFE) or other minimally permeable polymers.

The embodiments of the invention described above utilize generally “L” shaped punctum plugs 12, 12a, 12b wherein an elongate body portion 19 extends at a right angle to a substance insert receiving portion 20, 20a, 20b. It is to be appreciated, however, that the present invention may also be used in conjunction with any other punctum plug configurations and designs, many of which do not extend into the horizontal portion of the canaliculis LC. Examples of some alternative punctum plugs 12c, 12d and 12e that may be used in place of those described above are shown in FIGS. 8A, 8B and 8C as well as others disclosed in the patent applications listed in the above-set-forth table and expressly incorporated herein by reference

The devices and methods of this invention may be used to treat a wide variety of disorders as described in detail in the applications listed in the above-set-forth table and expressly incorporated herein by reference. In particular, the devices and methods of this invention may be used to deliver desired substance(s) to the anterior surface of the eye to treat an eye disorder that affects the cornea, conjunctiva, sclera, anterior chamber, trabecular meshwork, Schlemm's canal, collector channels emanating from Schlemm's canal or other anatomical structures in the eye (e.g., structures of the anterior segment) to which a therapeutically effective amount of a substance that has been administered topically to the anterior surface of the eye will distribute. Non-limiting examples of eye disorders that may be treated by therapeutic substance delivered using the devices and methods of this invention include glaucoma, ocular hypertension, corneal disorders, dry eye, allergies, infections, inflammations and pain.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

1. A substance delivery device that is implantable in a punctum of the eye of a human or animal subject, said device comprising:
a punctum plug comprising a plug body and a substance insert-receiving cavity formed in the plug body;
a substance insert comprising a substance eluting core that has a distal end and a proximal end;
the substance insert being positioned in the substance insert-receiving cavity of the punctum plug such that, when the device is implanted in the punctum of an eye;
a) a first tear-eluting location at a proximal end of the substance eluting core will be exposed to tears and/or other fluid(s) that distribute to the anterior surface of the eye so that the substance will elute from that first tear-eluting location into tears and/or other fluid(s) that distribute to the anterior surface of the eye; and
b) at least one additional tear-eluting location on the substance eluting core will also be exposed to tears and/or other fluid(s) that distribute to the anterior surface of the eye so that the substance will also elute from said at least one additional tear-eluting location into tears and/or other fluid(s) that distribute to the anterior surface of the eye.

2. A device according to claim 1 wherein the first tear-eluting location comprises the entire proximal end of the substance eluting core.

3. A device according to claim 1 wherein at least part of the distal end of the substance eluting core is impervious to the substance such that the substance is substantially prevented from eluting from the distal end of the substance eluting core.

4. (canceled)

5. A device according to claim 1 wherein at least one channel is formed in the side of the substance insert such that tears and/or other fluid(s) may enter the channel and wherein said at least one additional tear-eluting location on the substance eluting core is located adjacent to the at least one channel.

6. (canceled)

7. A device according to claim 1 wherein the substance eluting core comprises a polymer matrix combined with a quantity of the substance.

8. A device according to claim 7 wherein the polymer matrix comprises a silicone.

9-11. (canceled)

12. A device according to claim 1 wherein the substance insert consists essentially of the substance eluting core.

13. (canceled)

14. A device according to claim 1 wherein the substance insert comprises the substance eluting core in combination with a sheath member, wherein the sheath member has a side wall, a lumen, an open proximal end and a distal end and wherein the substance eluting core is disposed in the lumen of the sheath member.

15. (canceled)

16. A device according to claim 14 wherein at least part of the sheath member is formed of a material that is substantially impervious to the substance such that the substance is substantially prevented from eluting therethrough.

17. A device according to claim 16 wherein the sheath member is formed of at least one material selected from the group consisting of polyimids, polyethylenes and polyethylene terephthalates.

18. (canceled)

19. A device according to claim 14 wherein at least one channel exists to allow tears and/or other fluid(s) to flow adjacent to the sheath member and wherein said least one additional tear-eluting location comprises one or more apertures formed in the sheath member adjacent to the at least one channel so that the substance will elute from the substance eluting core, through said one or more apertures and into tears and/or other fluid(s) that have entered said at least one channel.

20. A device according to claim 19 wherein said one or more apertures comprise a plurality of apertures arranged in rows.

21. A device according to claim 20 wherein the substance insert further comprises least one marker useable to facilitate positioning of the substance insert within the substance insert-receiving cavity such that the rows of apertures are located adjacent to the channels.

22-29. (canceled)

30. A device according to claim 1 wherein the plug body is formed of at least one material selected from the group consisting of: elastomers, silicones, hydrophilic polymers, hydrophilic silicones, polyurethanes, acrylics, polyvinyl alcohol, hydrogels, polyurethane hydrogels, solid hydrogels, silicone/polyurethane copolymers, silicone/polyethylene glycol copolymers, silicone12 hydroxyethyl methacrylate copolymers (HCMA) any combination(s) thereof.

31. A device according to claim 1 wherein at least a portion of the plug body located adjacent to the substance insert-receiving cavity is impervious to the substance by a coating that is impervious to the substance, thereby preventing the substance from eluting through that portion of the plug body.

32-33. (canceled)

34. A device according to claim 31 wherein the coating is selected from the group consisting of: cyanoacrylates; UV curable cyanoacrylates; parylene (poly-xylylene polymers), metallic coatings; silver-based coatings; polyimides, impervious silicones, thermoplastics; polyurethane; polyvinyl chloride; polytetrafluoroethylene (PTFE), and any combination(s) thereof.

35-36. (canceled)

37. A device according to claim 1 wherein the device delivers a therapeutically effective amount of a substance to treat an eye disorder that affects the cornea, conjunctiva, sclera, anterior chamber, trabecular meshwork, Schlemm's canal, collector channels emanating from Schlemm's canal or other anatomical structures in the eye to which a therapeutically effective amount of a substance that has been administered topically to the anterior surface of the eye will distribute.

38. A device according to claim 37 wherein the device delivers a therapeutically effective amount of a substance to treat a disorder selected from the group consisting of:
glaucoma, ocular hypertension, corneal disorders, dry eye, allergies, infections, inflammations and pain.

39. (canceled)

40. A device according to claim 37 wherein the therapeutic substance is selected from the group consisting of: latanoprost, travaprost, and bimatoprost.

41. A method for treating a disorder of the eye in a human or non-human animal subject, said method comprising the steps of:
(A) obtaining a substance delivery device that is implantable in a punctum of the eye of a human or animal subject, said device comprising:
a punctum plug comprising a plug body and a substance insert-receiving cavity formed in the plug body;
a substance insert comprising a substance eluting core that has a distal end and a proximal end;
the substance insert being positioned in the substance insert-receiving cavity of the punctum plug such that, when the device is implanted in the punctum of an eye;
a) a first tear-eluting location at a proximal end of the substance eluting core will be exposed to tears and/or other fluid(s) that distribute to the anterior surface of the eye so that the substance will elute from that first tear-eluting location into tears and/or other fluid(s) that distribute to the anterior surface of the eye; and
b) at least one additional tear-eluting location on the substance eluting core will also be exposed to tears and/or other fluid(s) that distribute to the anterior surface of the eye so that the substance will also elute from said at least one additional tear-eluting location into tears and/or other fluid(s) that distribute to the anterior surface of the eye;
wherein the substance eluting core elutes a substance that is effective to treat the disorder;
(B) implanting the substance delivery device in the punctum of an eye of the subject such that the substance elutes from the substance core into tears and/or other fluid(s) that distributes to the anterior surface of the eye in a concentration that results in a delivery of a therapeutically effective dose of the substance to or through the cornea of the eye.

42. A method according to claim 41 wherein the disorder is glaucoma, and wherein the substance is a substance that is effective to treat glaucoma and wherein Step B results in transcorneal delivery of a therapeutically effective amount of the substance into the anterior chamber of the eye.

43. (canceled)
